(19) United States
(12) Patent Application Publication
(10) Pub. No.: US 2003/0198687 A1
(43) Pub. Date: Oct. 23, 2003

(54) Wound care composition

(75) Inventors: Keith Bennett, Hot Springs, AR (US); John H. Hartwig, Jamaica Plain, MA (US)

Correspondence Address: HAYNES AND BOONE, LLP, 901 MAIN STREET, SUITE 3100, DALLAS, TX, 75202, US

(73) Assignee: Keith Bennett, M.D., Hot Springs, AR

(21) Appl. No.: 10/125,676

(22) Filed: Apr. 18, 2002

(51) Int. Cl. A61K 35/14

(52) U.S. Cl. 424/532; 424/198.1; 424/78.06; 424/93.72

(57) ABSTRACT

A method and composition for wound care is provided, the composition comprising autologous platelet-rich plasma that has been contacted with an activator.

### BACKGROUND

The present embodiment relates generally to a method and composition for wound care.

Under conditions well known to those skilled in the art, intervention is required for healing certain types of wounds in mammals, often due to the condition of the mammal having the wound. One general type of wound care composition takes advantage of autologous blood, and portions of its inherent red blood cells, plasma, platelets, growth factors, and white blood cells. One such composition type that has been shown to be effective comprises relatively concentrated platelets (see U.S. Pat. Nos. 5,733,545 and 5,165,938, and U.S. Patent Publication No. 2001/0004638). However, such compositions and methods of using such compositions require isolating the platelets from both the red blood cells and the plasma and white blood cells. Thus, they are labor intensive and require a relatively large amount of blood from the mammal.

Therefore, what is needed is an autologous wound care method and composition that is less complicated, and therefore less expensive, and requires less blood from the mammal.

### BRIEF DESCRIPTION OF THE DRAWINGS

FIG. 1 is a perspective view with a broken away portion of an activation chamber according to one embodiment of the present invention.

### DESCRIPTION

A composition for wound care comprises autologous platelet-rich plasma and an activator. A method for wound care comprises preparing autologous platelet-rich plasma, mixing the autologous platelet-rich plasma with an activator, and applying the mixture to a wound. The term “wound” as used in this application refers to any type of wound resulting from conditions and events such as trauma, surgery, burns, ulcers, abrasions, donor sites, graft sites and cosmetic skin treatments such as laser skin resurfacing. The term “platelet-rich plasma” has a well known meaning in the medical arts, and its method of preparation is well established.

Preferably, autologous platelet-rich plasma is prepared by drawing blood from the mammal having the wound, centrifuging the blood, and drawing off the supernatant (platelet-rich plasma), which comprises plasma, white blood cells, and platelets. The amount of platelet-rich plasma required understandably depends on the size of the wound to be treated. However, 10 mL of platelet-rich plasma is a convenient amount to obtain, requiring approximately 20 mL of blood to be drawn. Thus, 15 to 45 mL of blood is normally drawn in a conventional manner from the mammal, and the blood is placed in a blue stopper top vacuum tube containing sodium citrate to prevent coagulation (available from BD (Becton, Dickinson and Company) under the trademark “VACUTAINER”).

The blood is centrifuged at speeds sufficient to produce forces of 135 to 280 g for 3 to 5 minutes at 20 to 37° C. The platelet-rich plasma is then drawn off to be used in the composition.

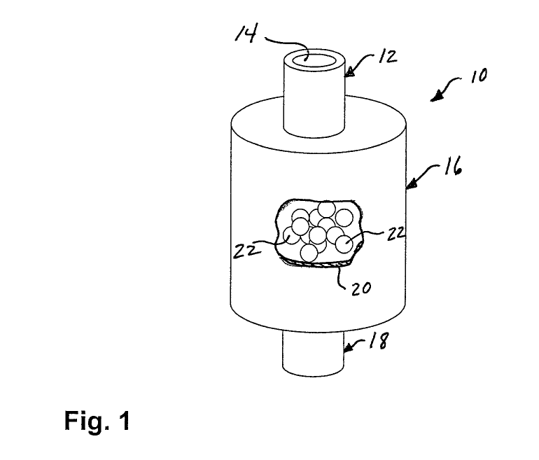

Activators refer to any known compound for activating the platelets in the platelet-rich plasma. The activators include thrombin, collagen, serotonin, acetylcholine (ACT), adenosine diphosphate (ADP), thrombin receptor-activating protein (TRAP; described in Hartwig, J., G. Bokoch, C. Carpenter, P. Janmey, L. Taylor, A. Toker, and T. Stossel, 1995, Thrombin receptor ligation and activated Rac uncap actin filament barbed ends through phosphoinositide synthesis in permeabilized human platelets, Cell 82:643-653; and Vu, T., D. Hung, V. Wheaton, and S. Coughlin, 1991, Molecular cloning of a functional thrombin receptor reveals a novel proteolytic mechanism for receptor activation, Cell 64:1057-1068; the entire disclosures of which are hereby incorporated by reference as if reproduced in their entirety, a peptide having an amino acid sequence of Ser-Phe-Leu-Leu-Arg-Glu or alternatively, Ser-Phe-Leu-Leu-Arg-Glu-Pro-Glu-AspLys-Tyr-Glu-Pro-Phe), or glass (as will be described with reference to FIG. 1). The amount of activator required to activate the platelets in the platelet-rich plasma varies with the activator and the amount of platelet-rich plasma to activate, but is easily determined, or may be achieved by step-wise addition of the activator in aliquots. Activation is done in the presence of greater than 100 μM calcium, such as calcium chloride.

In a preferred embodiment, the activator is TRAP, and preferably 0.2 to 0.4 micrograms of TRAP are mixed with 10 mL of platelet-rich plasma to result in a final concentration of 10-25 micromolar TRAP. In another preferred embodiment, the activator is a mixture of TRAP and ADP. In still another preferred embodiment, the activator is a composition that includes from 20 to 1000 units of bovine thrombin mixed in 1 mL of saline in which 0.5 mL of the thrombin-saline mixture is mixed in 1 mL of calcium chloride to produce a composition that includes approximately 13.6 milliequivalents of calcium per 10 mL of the activator composition.

In a second preferred embodiment, the activator is glass. Referring to FIG. 1, an activator chamber 10 has an inlet 12 having an opening 14. A chamber 16 is in fluid communication with the inlet 12, and with an outlet 18. The inlet 12 and outlet 18 may be adapted to receive conventional syringes or needles (not depicted). For example, the outlet 18 may have a luer lock feature to attach to a blunt needle.

The chamber 16 has a wall 20, defining a substantially hollow interior portion. The wall 20 may be transparent to allow viewing of the interior portion. In one embodiment, the interior portion has a volume of approximately 1 to 5 cubic centimeters.

A plurality of glass beads 22 are disposed in the chamber 16. The size of the glass beads preferably ranges from 1 to 3 mm in diameter, and suitable glass beads are available from Fisher Scientific, VWR International, PGC Scientifics Corporation or Erie Scientific Company. The chamber 16 includes conventional means (not depicted), such as screens, for retaining the glass beads 22 in the chamber, while allowing fluid to flow between the inlet 12 and the outlet 18. The glass beads 22 are depicted for illustrative purposes, however, all conventional means to present a surface area of glass to the fluid are contemplated.

In operation, a syringe containing platelet-rich plasma is placed at the inlet 12, and the platelet-rich plasma flows into the chamber 16. The glass beads 22 activate the platelets in the platelet-rich plasma, and the activated platelet-rich plasma is removed from the chamber 16 via the outlet 18, and used as a wound care composition.

In an alternative embodiment of the wound care composition, the composition may further comprise a matrix material for thickening the composition, for example a calcium salt of alginic acid, available from Maersk Medical, Denmark under the trademark “SORBSAN,” or collagen available from Biocore Medical Technologies, Inc., Topeka, Kans., under the trademark “KOLLAGEN.”

In another alternative embodiment of the wound care composition, the composition may further comprise increased levels of growth factor, for example the composition may be used during surgery, such as in bone grafting procedures, with increased levels of insulin-like growth factor (IGF). Likewise, as the composition increases blood flow, it may be used with increased levels of vascular endothelial growth factor (VEGF) to promote hair growth. It is believed that since both growth factors naturally occur in blood, the first-described wound care composition would be efficacious for these uses even without increased levels of growth factor.

The following examples are illustrative of the methods and compositions discussed above.

### EXAMPLE 1

A wound care composition was prepared from an individual afflicted with chronic diabetic foot ulcer having a diameter of 4 cm. An amount of whole blood (approximately 20 mL) sufficient to obtain approximately 10 mL of platelet-rich plasma was drawn using a butterfly needle and blue stopper-topped vacuum tubes. The blood was centrifuged at approximately 135 g for 1 minute at approximately 20° C., and the platelet-rich plasma obtained. The platelets were activated by the addition of 1 mL of an activator composition to 10 mL of the platelet-rich plasma. According to this example the activator composition was selected from 25 micromolar of TRAP or 10 micromolar of ADP or a mixture of 25 micromolar of TRAP and 10 micromolar of ADP.

It is of note that the composition was a liquid at this point. To obtain a gel, the liquid composition can be poured into an uncoated glass Petri dish. Under those conditions, the liquid composition solidifies to form a gel in approximately less than one minute.

The wound was completely debrided, and the composition was applied to the wound on a gauze bandage, and covered with an occlusive dressing. After five to seven days, the wound was cleansed with saline, debrided and an alternate dressing was applied until the next treatment. At the time of the next treatment, a second batch of the wound care composition prepared as described above was applied directly to the wound or on a gauze bandage, and covered with an occlusive dressing. Successive batches of composition were applied every seven to fourteen days, until the wound was completely healed, approximately six weeks after the first treatment.

### EXAMPLE 2

A wound care composition can be prepared from an individual afflicted with a chronic wound. An amount of whole blood (approximately 20 mL) sufficient to obtain approximately 10 mL of platelet-rich plasma can be drawn using a butterfly needle and blue stopper-topped vacuum tubes. The blood can be centrifuged at 135 to 280 g for 3 to 5 minutes at 20 to 37° C., and the platelet-rich plasma obtained. 2 to 4 microgram (μg) of TRAP can be added to 10 mL of the platelet-rich plasma to activate the platelets.

The wound should be completely debrided, and the composition applied to the wound, and covered with an occlusive dressing. After five to seven days, the wound should be cleansed with saline, debrided and an alternate dressing applied until the next treatment. At the time of the next treatment, a second batch of the wound care composition prepared as described above should be applied directly to the wound or on a gauze bandage, and covered with an occlusive dressing. Successive batches of composition can be applied every seven to fourteen days, until the wound is completely healed.

### EXAMPLE 3

A wound care composition can be prepared from an individual afflicted with a chronic wound. An amount of whole blood (approximately 20 mL) sufficient to obtain approximately 10 mL of platelet-rich plasma can be drawn using a butterfly needle and blue stopper-topped vacuum tubes. The blood can be centrifuged at 135 to 280 g for 3 to 5 minutes at 20 to 37° C., and the platelet-rich plasma obtained. 10 mL of the platelet-rich plasma can be passed through the activator chamber (FIG. 1) to activate the platelets.

The wound should be completely debrided, and the composition applied to the wound, and covered with an occlusive dressing. After five to seven days, the wound should be cleansed with saline, debrided and an alternate dressing applied until the next treatment. At the time of the next treatment, a second batch of the wound care composition prepared as described above should be applied directly to the wound or on a gauze bandage, and covered with an occlusive dressing. Successive batches of composition can be applied every seven to fourteen days, until the wound is completely healed.

### EXAMPLE 4

A wound care composition can be prepared from an individual afflicted with a chronic wound. An amount of whole blood (approximately 20 mL) sufficient to obtain approximately 10 mL of platelet-rich plasma can be drawn using a butterfly needle and blue stopper-topped vacuum tubes. The blood can be centrifuged at 135 to 280 g for 3 to 5 minutes at 20 to 37° C., and the platelet-rich plasma obtained. 10 mL of the platelet-rich plasma can be activated in any of the above-described manners to activate the platelets. Next, a matrix material for thickening the composition can be added, for example, a sufficient amount of SORBSAN a calcium salt of alginic acid available from Maersk Medical, A/S, to fill in the wound.

The wound should be completely debrided, and the composition applied to the wound, and covered with an occlusive dressing. After five to seven days, the wound should be cleansed with saline, debrided and an alternate dressing applied until the next treatment. At the time of the next treatment, a second batch of the wound care composition prepared as described above should be applied directly to the wound or on a gauze bandage, and covered with an occlusive dressing. Successive batches of composition can be applied every seven to fourteen days, until the wound is completely healed.

### EXAMPLE 5

A wound care composition can be prepared from an individual afflicted with a chronic wound. An amount of whole blood (approximately 20 mL) sufficient to obtain approximately 10 mL of platelet-rich plasma can be drawn using a butterfly needle and blue stopper-topped vacuum tubes. The blood can be centrifuged at 135 to 280 g for 3 to 5 minutes at 20 to 37° C., and the platelet-rich plasma obtained. 10 mL of the platelet-rich plasma can be activated in any of the above-described manners to activate the platelets. Next, a matrix material for thickening the composition can be added, for example, a sufficient amount of KOLLAGEN collagen available from Biocore Medical Technologies, Inc., to fill in the wound.

The wound should be completely debrided, and the composition applied to the wound, and covered with an occlusive dressing. After five to seven days, the wound should be cleansed with saline, debrided and an alternate dressing applied until the next treatment. At the time of the next treatment, a second batch of the wound care composition prepared as described above should be applied directly to the wound or on a gauze bandage, and covered with an occlusive dressing. Successive batches of composition can be applied every seven to fourteen days, until the wound is completely healed.

### EXAMPLE 6

A wound care composition can be prepared from an individual undergoing a bone grafting procedure. An amount of whole blood (approximately 20 mL) sufficient to obtain approximately 10 mL of platelet-rich plasma can be drawn using a butterfly needle and blue stopper-topped vacuum tubes. The blood can be centrifuged at 135 to 280 g for 3 to 5 minutes at 20 to 37° C., and the platelet-rich plasma obtained. 10 mL of the platelet-rich plasma can be activated in any of the above-described manners to activate the platelets. Alternatively, additional IGF can be added.

The composition should be applied all around the graft or alternatively the bone chips or fragments may be mixed with the platelet gel prior to or during implantation.

### EXAMPLE 8

A wound care composition can be prepared from an individual to promote hair growth. An amount of whole blood (approximately 20 mL) sufficient to obtain approximately 10 mL of platelet-rich plasma can be drawn using a butterfly needle and blue stopper-topped vacuum tubes. The blood can be centrifuged at 135 to 280 g for 3 to 5 minutes at 20 to 37° C., and the platelet-rich plasma obtained. 10 mL of the platelet-rich plasma can be activated in any of the above-described manners to activate the platelets. Alternatively, additional VEGF can be added.

Approximately every two weeks, the composition should be injected into the subcutaneous tissue where hair growth is desired.

Although only a few exemplary embodiments of this invention have been described in detail above, those skilled in the art will readily appreciate that many other modifications are possible in the exemplary embodiments without materially departing from the novel teachings and advantages of this invention. For instance, while the present wound care compositions and methods have been describe in the context of treating humans, these wound care compositions and methods can also be useful in veterinary applications. Accordingly, all such modifications are intended to be included within the scope of this invention as defined in the following claims.

1. A method of treating a wound on a mammal comprising:
preparing a wound care composition comprising autologous platelet-rich plasma;
contacting the autologous platelet-rich plasma with an activator; and
placing the wound care composition on the wound.

2. The method of claim 1 wherein the activator comprises a source of calcium and at least one member selected from the group consisting of: thrombin, collagen, serotonin, acetylcholine, or adenosine diphosphate.

3. The method of claim 1 wherein the activator comprises thrombin receptor-activating protein.

4. The method of claim 1 wherein the activator is glass.

5. The method of claim 1 further comprising adding a matrix material to the composition.

6. The method of claim 1 further comprising passing the platelet-rich plasma through an activation chamber.

7. The method of claim 1 further comprising adding a growth factor to the composition.

8. A wound care composition for treating a wound on a mammal comprising:
autologous platelet-rich plasma that has been contacted with an activator.

9. The composition of claim 8 wherein the activator comprises a source of calcium and at least one member selected from the group consisting of: thrombin, collagen, serotonin, acetylcholine, or adenosine diphosphate.

10. The composition of claim 8 wherein the activator comprises thrombin receptor-activating protein.

11. The composition of claim 8 wherein the activator is glass.

12. The composition of claim 8 further comprising a matrix material.

13. The composition of claim 8 further comprising a growth factor.

14. An activation chamber for activating the platelets in platelet-rich plasma, comprising:
a chamber having an inlet and an outlet for passing the platelet-rich plasma through the chamber,
wherein the chamber has a surface area of glass for contacting the platelet-rich plasma, thereby activating the platelets.

15. The activation chamber of claim 14 wherein the surface area of glass is provided by glass beads.

16. The activation chamber of claim 14 wherein the outlet has a luer lock feature.

17. A method of treating a mammal comprising:
drawing blood from the mammal;
centrifuging the blood to obtain autologous platelet-rich plasma;
activating the autologous platelet-rich plasma; and
placing the activated autologous platelet-rich plasma on the mammal.

18. The method of claim 17 wherein the mammal is treated to close a wound.

19. The method of claim 17 wherein the mammal is treated to speed healing from a bone grafting procedure.

20. The method of claim 17 wherein the mammal is treated to promote hair growth.
